(19) United States
(12) Patent Application Publication
(10) Pub. No.: US 2021/0236541 A1
(43) Pub. Date: Aug. 5, 2021

(54) IODOPHOR COMPOSITION WITH IMPROVED STABILITY IN THE PRESENCE OF ORGANIC MATERIAL

(71) Applicant: National University of Ireland, Galway, Galway (IE)

(72) Inventors: Vincent O'FLAHERTY, Galway (IE); Paul MC CAY, Galway (IE)

(21) Appl. No.: 17/176,782

(22) Filed: Feb. 16, 2021

Related U.S. Application Data
(62) Division of application No. 15/504,616, filed on Feb. 16, 2017.

(30) Foreign Application Priority Data
Aug. 20, 2014 (EP) 14181684.3

(51) Int. Cl. A61K 33/18; A23C 3/08; A23C 7/02; A01N 59/12; A61K 9/00; A61K 9/06; A61K 47/02; A01N 25/22; A61K 47/20; A61K 9/70; A61K 47/59; A61K 8/20; A61Q 11/00

(52) U.S. Cl. CPC A61K 33/18; A61Q 11/00; A23C 7/02; A01N 59/12; A61K 9/0078; A61K 9/06; A61K 47/02; A01N 25/22; A61K 9/0041; A61K 47/20; A61K 9/0014; A61K 9/7007; A61K 47/59; A61K 8/20; A61K 9/0043; A23C 3/08

(57) ABSTRACT

The invention is concerned with a pharmaceutical and industrial iodophor preparation, its synthesis and potential applications. The compound has predictable antimicrobial activities. Furthermore, this iodophor is much more stable in the presence of organic material than traditional iodophors. The compositions release free iodine when in solution, which provides the antimicrobial activity.

### BACKGROUND OF THE INVENTION

This invention is concerned with a pharmaceutical and industrial iodophor preparation, its synthesis and potential applications. The compound has predictable antimicrobial activities. Furthermore, this iodophor is much more stable in the presence of organic material than traditional iodophors.

Iodophors are iodine based compounds complexed with a stabilization agent. They release free iodine when in solution, which provides the antimicrobial activity. They are typically prepared by mixing iodine with the stabilization agent, with heating.

Iodophors exert antimicrobial activity as the free iodine interacts with key bacterial proteins causing bacteriostatic or bacteriocidal effects. They have a potential wide-range of uses, from cleaning and sterilising industrial or medical equipment, to wound treatment (after or before infection as a prophylactic). Diluted iodophors are used extensively in the dairy industry to clean machinery, minimising the spread of potential pathogenic organisms. Iodophors are not only antimicrobial but strongly antiviral and antifungal. Their use requires very little post-rinsing, unlike hypochlorite based bleaching compounds. Their use is common in aquatic tanks as a method of minimising excess growth of bacteria or parasitic organisms. They can be used to kill causative organisms associated with acne and skin conditions, as well as a treatment for the flare up of herpes infections (virus). Iodophors have been used for many years and are generally recognised as safe compounds. Based on a review of the available toxicology data, the US EPA concluded that “iodine and iodophor complexes are of very low toxicity by the oral, dermal, and inhalation routes of exposure”. The most common iodophor is polyvinylpyrrolidone iodine (known as PVP-iodine, PVP-I, or povidone, and by its brand name Betadine, or Wokadine). The PVP polymer also serves to reduce the potential toxicity effects of iodine. There have been very few reported resistance/tolerance characteristics in bacteria to iodine-mediated killing. This is more than likely due to the indiscriminate targeting by the iodophor of various bacterial proteins within the cell. Such tolerances/resistance phenotypes would require multiple, and simultaneous beneficial mutations in the bacterial genome.

The use of PVP-I has overcome some of the disadvantages of tinctures of iodine and Lugol's solution (a solution of iodine with iodide ions) as regards long term storage, free vs total iodine and toxicity issues etc. However, the commonly used iodophors such as PVP-I are very unstable in the presence of organic material. On dissolving PVP-I in water, the compound exerts significant antimicrobial activity, but minimal antimicrobial activity in the presence of organic matter. For example, the minimum inhibitory concentration (MIC) of PVP-I in water for E. coli ATCC 25922 is <1 mg L⁻¹. On repetition of the test in an organic rich growth medium (such as lysogeny broth), the MBC value is recorded at >500 mg L⁻¹. This discrepancy in MBC values indicates the limitations of traditional iodophors. Use of such a concentration in many environments would be practically difficult, prohibitively expensive and potentially toxic.

In the dairy industry, PVP-I is regularly used to clean teats of cows before and after milking to kill bacteria present. However, if the PVP-I comes into contact with milk, activity is quickly diminished. The same pattern occurs in other industries, such as baking etc, as food residue left on machinery can interact with the PVP-I and halt its antimicrobial effect.

### OBJECT OF THE INVENTION

An object of the present invention is to provide a novel iodophor which is considerably more stable in the presence of organic materials than traditional iodophors. A further object is to provide iodophor producing compositions, particularly compositions which can be used for sterilising environments with a high organic matter load.

### SUMMARY OF THE INVENTION

According to the present invention there is provided an iodophor-producing composition comprising iodide or iodate ions, thiocyanate ions and an oxidising agent.

The source of the iodide/iodate may be potassium iodide, sodium iodide, lithium iodide, caesium iodide, hydrogen iodide, rhodium iodide, or a slow-release form of iodide, potassium iodate or sodium iodate.

The source of the thiocyanate ions may be potassium thiocyanate, lithium thiocyanate, caesium thiocyanate, hydrogen thiocyanate, rhodium thiocyanate, a slow-release form of thiocyanate, or a cyanate compound such as potassium cyanate or thiocyanogens.

The oxidising agent may be hydrogen peroxide or potassium permanganate, sodium peroxide, lithium peroxide, peroxide releasing percarbonates, peroxide releasing citric acid or vitamin C, peroxide salts including barium oxide, sodium perborate, a hydrogen peroxide-urea adduct, oxygen releasing pseudo-peroxides including superoxides, dioygenals, ozones, ozonides, organic peroxides including peroxy acids, acyl halides and aliphatic peroxides.

In some embodiments potassium iodate may serve as both the oxidising agent and the source of the iodate/iodine ions.

The invention also provides an iodophor comprising an Iodo-thiocyanate complex containing an oxygen, such as I₂OHS(CN)₂.

The novel iodophor can be prepared by the mixing of a source of iodide ions (such as potassium iodide), a source of thiocyanate ions (such as potassium thiocyanate) and an oxidising agent, such as hydrogen peroxide in an aqueous environment. A strongly antimicrobial compound can be prepared by the mixing of 1% w/v potassium thiocyanate (KSCN), 1% w/v potassium iodide (KI), and 1% w/v hydrogen peroxide (H₂O₂). More dilute or concentrated solutions of iodophor can be prepared by the increase or decrease in starting components. Likewise, a concentrated stock can be prepared, and diluted accordingly when required.

In this aqueous environment, the iodide and thiocyanate are simultaneously oxidised, and form an iodo-thiocyanate complex containing an oxygen atom. Further oxidation can occur, producing I₂, I₃, I₄, and I₅ species, increasing in molecular weight.

Use of hydrogen peroxide is advantageous in that there are no significant breakdown or toxicity issues as it is cleaved to form oxygen and water. Other oxidising agents can be used, for example potassium permanganate, though they do add unwanted colour and impurities to the environment. Alternatively, potassium iodate (KIO₃, 1% w/v) can serve as both the oxidising agent and iodine source as an alternative to the combination of peroxide and iodide. The reaction between it and thiocyanate proceeds at a slower rate (taking almost 24 hours at the concentrations described herein, though the reaction can be quickened up by addition of hydrogen peroxide). Importantly the iodate based reaction requires a concentration above 1% w/v of the components to produce significant antimicrobial activity. Using the iodide/thiocyanate/peroxide model, the solution can be made using much lower (0.001-0.01% w/v) or higher concentrations and the antimicrobial activity exerted will have a linear relationship to concentration, unlike the iodate model. This is surprising. The iodophor, if prepared in an aqueous environment, can be buffered to a pH if required. It is stable in the presence of numerous salts, and at a relatively large temperature range if in a sealed environment. The iodophor can be prepared as a hydrogel (using for example 1% w/v sodium polyacrylate). This lends itself to use as a gel for burn/skin complaints.

A 5-20 ml volume of 0.5-2% w/v prepared iodophor would be capable of infusion into the udder of a mastitic ruminant animal and killing the causative organism. The treatment regimen could mirror that of antibiotic treatments (once or twice a day, for 2-10 days for a mastitic animal).

Because of the wide-ranging antimicrobial activity spectrum exerted by iodophors, the compound would be capable of killing a wide variety of bacterial (including Gram-positive, Gram-negative, antibiotic-resistant bacteria, fungal strains, and viruses).

Dilute forms (<0. 1% w/v) of the novel iodophor could be used to treat minor cuts or abrasions. More concentrated forms of the same iodophor could be used to treat more serious skin infections (˜0.5% w/v). A nebulised spray version of the iodophor may be suitable for use to treat lung infections where antibiotic usage is commonly unsuccessful. Such a delivery mechanism would also have a potential in surgeries where open wounds occur as a means of prophylactic.

Concentrated forms (>1% w/v) could be used to clean/sterilise or decontaminate inanimate materials such as food processing equipment, steel bowls, surgical equipment etc.

Although it is preferential that simple ionic salts such as potassium or sodium variants of iodide and thiocyanate be used, other forms may also be used including, but not limited to sodium iodide, potassium iodide, lithium iodide, caesium iodide, hydrogen iodide, rhodium iodide, or by the oxidation of elemental iodine and as a source of thiocyanate; potassium thiocyanate, lithium thiocyanate, caesium thiocyanate, hydrogen thiocyanate, rhodium thiocyanate, or a cyanate compound such as potassium cyanate or thiocyanogens.

Likewise, the use of hydrogen peroxide as an oxidising solution is preferable as it is non-toxic at required concentrations (<3% w/v) and leaves no significant residues. Alternative, less preferential, oxidising agents may be used including, but not limited to sodium peroxide, lithium peroxide or peroxide releasing percarbonates, peroxide releasing citric acid or Vitamin C, peroxide salts including barium oxide, sodium perborate, an hydrogen peroxide-urea adduct, oxygen releasing pseudo peroxides including superoxides, dioygenals, ozones, and ozonides, organic peroxides including peroxy acids, acyl halides, aliphatic peroxides.

The iodophor can be prepared as a gel, an emulsion, an oil, a hydrogel, a liquid or a powder as suits the use. The compound can be lyophilized and stored as a dried version to prolong storage. The compound can be added to bandages as a dried (and subsequently wetted) or wet product as a means to prevent or kill bacterial infection. The compound can be adapted for oral delivery, intravenous delivery, topical delivery, enteral or parenteral delivery, or delivery by inhalation. The compound may be formulated together with conventional pharmaceutically acceptable carriers and excipients. Suitable carriers and excipients include, but are not limited to, binders, coatings, colours, disintegrants, flavours, anti-adherents, glidants, lubricants, preservatives, sorbants, sweeteners or vehicles.

A ratio range of 0.1:10-10:1 of iodide to thiocyanate (by weight) is important in producing significant quantities of free available iodine with the oxygenated thiocyanate stabiliser. A more preferable ratio is 0.2:1-5:1. A more preferable ratio is 0.8:1-1.2:1. A more preferable option again is a 1:1 ratio of thiocyanate to iodide. For example; 1,000 mg L⁻¹ each of potassium iodide and potassium thiocyanate, in the presence of sufficient hydrogen peroxide acting as an oxidising agent 800-1200 mg L⁻¹.

A ratio range of 0.25:1-4:1 of hydrogen peroxide to iodide/thiocyanate (by weight) is sufficient in allowing the simultaneous oxidation and thus production of the iodophor. A more preferable option of hydrogen peroxide to iodide/thiocyanate is 0.5:1-2:1. A more preferable option again is a 1:1 ratio of hydrogen peroxide to iodide/thiocyanate. At ratios of 2:1 or greater of hydrogen peroxide to iodide/thiocyanate, the compound is less stable due to pH changes, and can result in the formation of undesirable CN and SO₄ as the thiocyanate SCN bonds are broken. At a range of <1:1 of hydrogen peroxide to iodide/thiocyanate, activity is reduced as insufficient oxidation occurs.

Percarbonates are desiccated compounds that release hydrogen peroxide on mixing with an aqueous environment. As such, sodium (and other) percarbonates could act as a source of hydrogen peroxide. A simple calculation based on the available hydrogen peroxide from the percarbonate would allow a simple swap of the liquid peroxide for a dried percarbonate. Furthermore, other compounds that oxidise and release free 0 could be used on determination of the free 0 content likely to be achieved. Simple oxidoreductase enzymes are capable of reacting with monosaccharide sugars to produce a source of hydrogen peroxide. Such a system could be used by titreing the correct level of hydrogen peroxide produced in the environment.

It is surprising that the use of a thiocyanate provides such stabilisation in the presence of organic material. SCN is a linear compound. It isn't one particular atom providing the basis for the characterisation. Allyl isothiocyanate was inefficient in producing the same results suggesting that the reaction is very dependent on access to the atoms. A number of sulfur (5) containing compounds including a variety of sulfates, thiols, and thio-based compounds such as methionine did not allow production of an organic-material-stable iodophor in the same manner. Likewise, a number of cyanate-based compounds such as sodium dichloroisocyanturate or potassium cyanate were not efficient in allowing the production of an organic-material-stable iodophor either. This makes the result of using thiocyanate all the more surprising and novel Depending on the degree of oxidation and the relative concentrations, a number of potential iodo-thiocyanate compounds are produced and released, from single iodine, right up to seven iodine atoms per molecule. Such compounds are ISCN, I₂SCN, I₂(SCN)₂, IOH(SCN)₂, I₃OH(SCN)₂, I₃OH(SCN)₃, I₄(SCN)₄OH, I₅(SCN)₅.

The iodophor of the invention is produced in solution by the simultaneous oxidation of iodide/iodate ions (to allow formation of iodine) and thiocyanate ions, which serve to stabilise the resulting antimicrobially active iodine. The resulting iodophor is an iodo-thiocyanate complex class compound. It can be prepared as a dilute or more concentrated preparation depending on requirements. The novel iodophor can be prepared in situ by the mixing of iodide/iodate and thiocyanate ions (in the presence of a suitable oxidising agent, for example hydrogen peroxide), or be prepared ex situ and applied/added accordingly. It exerts strong antimicrobial activity in water based environments like other iodophors. However, the MIC for E. coli ATCC25922 in lysogeny broth (LB) remains below 10 mg L⁻¹, unlike that for PVP-I (500 mg L⁻¹).

This novel iodophor has many potential uses as an antimicrobial agent where traditional iodophors have been used, namely; wound/burn treatment, an anti-viral topical treatment, machinery sterilant, skin wipe, anti-fungal topical agent. Furthermore, the novel iodophor is suited to a number of potential uses that traditional iodophor compounds would be unsuited for due to the presence of organic material. Examples of such environments include as a mastitis treatment for ruminant animals where the compound is used an alternative to antibiotics in the udder with milk present. A further example is for use to ‘clean’ untreated milk for eradication of the causative organism of Johne's disease, which can be transferred from mother to calf in the milk. The novel iodophor could be used to extend the shelf-life of milk in areas without sufficient refrigeration. This would not be possible with traditional iodophors due to quick dissipation of the antimicrobial activity on contact with milk. Furthermore, the novel iodophor is more suited to traditional applications as it would be less susceptible to dissipation by any present organic material (sloughed cells in a wound, organic debris on milking machinery etc. In addition, the iodophor has potential use as a replacement for antibiotic treatment, either topically, orally or intravenously. It could be used as a skin disinfectant, pre-operative skin preparation, topical application, treatment of the eye, treatment of the ear, acne treatment, treatment of viral infections or for use in dental settings e.g. as an anti-caries or anti-plaque rinse, as a pre-procedural rinse or in the sterilisation of endodontics. The compound also finds use in food sterilisation preparations; or in environmental applications e.g. sterilisation of surfaces, pipes; sterilisation of water (such as the impregnation of filters etc with the compound). The compound has the potential to replace the use of Povidone Iodine to which it is superior in activity.

The iodophor also may be used in cancer treatments where it could be used to replace the use of molecular iodine I₂ in for example breast cancers (Anguiano et al. Thyroid. Vol 17, No. 9, 2007, p 851-859).

### BRIEF DESCRIPTION OF THE DRAWINGS

FIG. 1: Effect of Lugol's solution and novel iodophor on milk colour (doubling dilutions left to right starting with 500 mg L⁻¹).

FIG. 2 Time-of-flight mass spectrometry (TOF-MS) analysis of 1% (1:1). m/z peaks were observable at 212, 232, 309, 386, 426, 483, 580, 669, 746, 796, 843, and 959.

FIG. 3; set-up of nebulisation simulation.

FIG. 4: set-up of nebulisation simulation.

FIG. 5: Kill efficiency of nebulised compound against E. coli.

FIG. 6: Kill efficiency of nebulised compound against P. aeruginosa.

### DETAILED DESCRIPTION OF THE INVENTION

### Examples of Compositions

1 A general wash/antimicrobial composition containing 1-100,000 mg L⁻¹ of the iodophor for use as an antimicrobial topical ointment/cream/gel/solution.
2 A general wash/antimicrobial composition containing 10-10,000 mg L⁻¹ of the iodophor for use as an antimicrobial topical ointment/cream/gel/solution.
3 A composition containing 10-10,000 mg L⁻¹ of the iodophor for use as an inhaled antimicrobial therapy using a nebulised solution
4 A bandage dipped in or containing 10-10,000 mg L⁻¹ of the iodophor for use as an antimicrobial bandage.
5 A composition for the treatment of mastitis in ruminant animals containing 10-10,000 mg L⁻¹ of the iodophor as an intra-mammary infusion for once or twice daily treatment
6 An extemporaneous antimicrobial composition for daily, or twice daily, treatment is made by means of intra-mammary devices containing components capable of producing 10-10,000 mg L⁻¹ of iodophor in situ as described above (iodide or iodate anion at between 10-10,000 mg L⁻¹, thiocyanate ion at between 10-10,000 mg L⁻¹ and an oxidising solution (if required) such as hydrogen peroxide at between 10-1,000 mg L⁻¹).
7 An extemporaneous antimicrobial (and anti-biofilm) composition general wash containing components capable of producing 10-1,000 mg L⁻¹ of iodophor in situ as described above (iodide or iodate anion at between 10-10,000 mg L⁻¹, thiocyanate ion at between 10-10,000 mg L⁻¹ and an oxidising solution (if required) such as hydrogen peroxide at between 10-10,000 mg L⁻¹) for any purposes as described in previous examples.
8 A composition as described in examples 1-7 wherein the oxidising solution is selected from a list described elsewhere in this text including, but not limited to, hydrogen peroxide, and present in sufficient concentrations to allow oxidation of the thiocyanate and iodide (or iodate) ions.

The pharmaceutically effective carrier is water, saline, an emulsion, a gel or a hydrogel.

The composition may be adapted for delivery by means of a dampened bandage.

The composition may be adapted for use as an antimicrobial nasal rinse, as an antimicrobial mouth-wash, an antifungal wash, as a disinfectant, added to a bandage or poultice for the treatment of wound or burn infections, or nebulised in the form of a spray for the treatment of bacterial or fungal infection of the human or animal lung.

The invention also provides a medical device coated with the compositions as described above, and a bandage or poultice impregnated with the compositions.

A composition described in any previous examples wherein it is supplemented to contaminated milk as an antimicrobial agent at concentrations of 10-10,000 mg L⁻¹ for the eradication of bacteria such as Mycobacterium avium paratuberculosis.

A composition described in any previous examples wherein it is supplemented to contaminated environments as an antimicrobial agent at concentrations of 10-10,000 mg L⁻¹ for the eradication of fungi, parasites, unicellular organisms, viruses, or bacterial/fungal spores.

A more stable form of the iodo-thiocyanate iodophor could be produced with the introduction of PVP polymer to the compound. Importantly, mixing thiocyanate with an iodine-containing PVP-I did not serve to change the structure of the existing PVP-I, demonstrating that the novel iodophor cannot be prepared in this manner.

Vince: give example of Paul's work with the egg dipping as an example of food prep usage?

### Example 9

A 1 L batch of iodophor is prepared by the addition of 10 g potassium iodide, 10 g potassium thiocyanate to ca. 950 ml of water. When the salts have dissolved fully, 33.33 ml of 30% hydrogen peroxide is added to the solution. The solution is then topped up to 1,000 ml with water. The solution can be pH adjusted and buffered to 5.5 if necessary. This solution is henceforth referred to as 1% (1:1) mixture, the ratio referring to the weight of the peroxide added to the weights of thiocyanate and iodide salts used.

An alternative to the use of hydrogen peroxide is to use ca. 33 g of sodium percarbonate (with 30% available hydrogen peroxide) and allowed dissolve fully to permit release of peroxide.

As a further alternative, a 1 L batch of the iodophor is prepared by the addition of 10 g potassium iodate and 10 g potassium thiocyanate to ca. 950 ml. The solution is then topped up to 1,000 ml with water and left for 24 hours to allow the reaction to occur. The solution can then be pH adjusted and buffered to 5.5 if necessary.

The novel iodophor could further be complexed with PVP polymer such that it would possess the advantages of both PVP-I in addition to the stability in the presence of organic material of the iodophor described in the examples.

A suitable glucocorticoid could be supplemented to intramammary infusions destined for mastitic animals, or in nebulised/general wash form destined for the treatment or prevention of lung infections to help with potential local tolerance issues. The steroid could be (but not limited to) hydrocortisone/cortisol, prednisolone, or prednisone present at between 5 and 50 mg per dose.

### Example 10

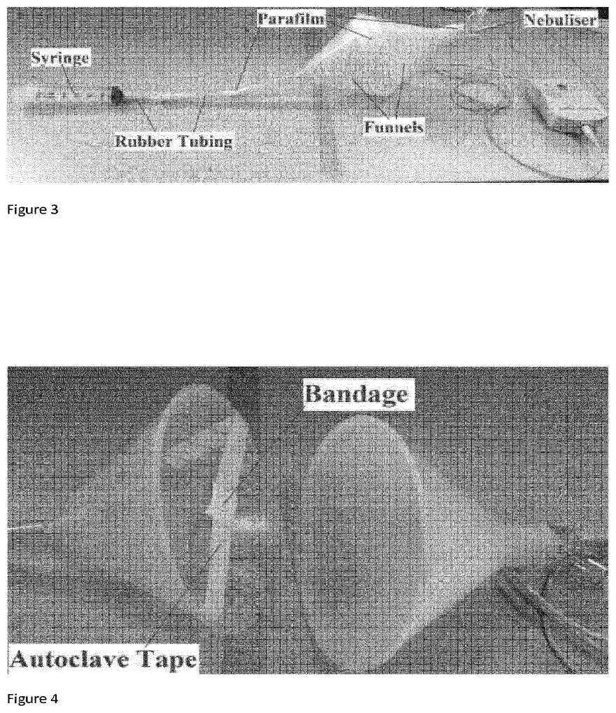

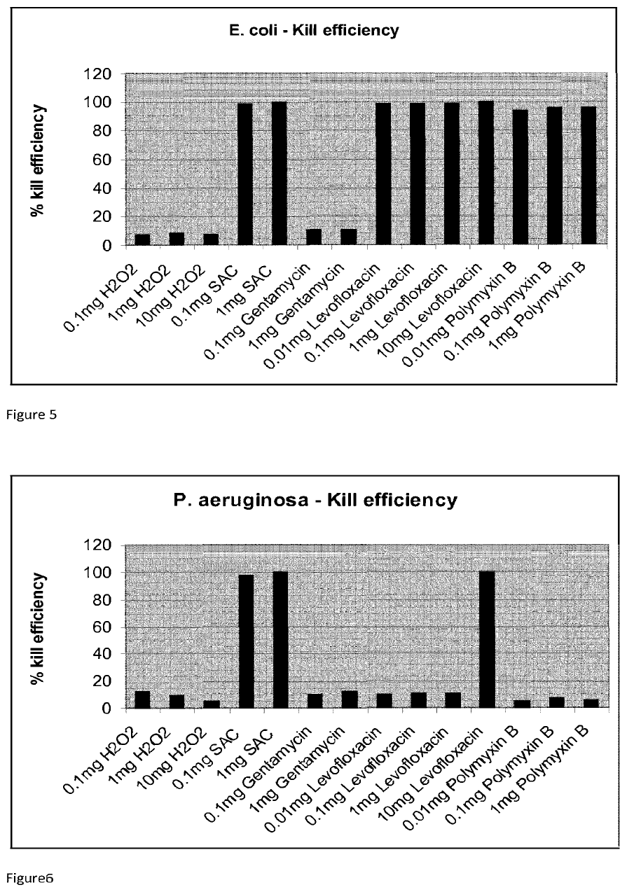

Two funnels, a 60 mL Syringe, rubber tubing, and a nebuliser were assembled as according to FIG. 3. A bandage piece was suspended in the middle of the two funnels, by placing a piece of gauze on a length of tape and sticking the bandage over the gauze as according to FIG. 4. 500 μL of the target bacteria (either E. coli ATCC 25922 or P. aeruginosa) was applied onto the bandage piece using an autopipette with a sterile tip. Both funnels were closed together, forming an area volume of 790 mL (while syringe was extended), and parafilm was applied around their rim in order to seal the apparatus shut and to keep the environment inside air tight. 1 mL of the compound of choice was placed into the nebuliser chamber, the chamber was then closed. The nebuliser (Aeroneb Pro-X Solo Nebuliser System manufactured by Aerogen Ltd.) was switched on to begin the aerosolisation of the compound, the inoculated bandage was then exposed to 1 mL of aerosolised compound in the chamber for 15 minutes. After 15 minutes of exposure to the compound, the inoculated bandage was suspend in 10 mL of 1% PBS solution, in a sterile universal tube. In between applying each compound, the nebuliser was cleaned by passing 1 mL of sterile H₂O through it. The tube was shaken and placed on the vortex in order to release bacterial cells contained in the bandage into solution. A 20 μL sample was taken from the PBS-E. coli solution and using a 96-well plate, 1 in 10 dilutions were performed until a dilution of 10-4 was achieved. 20 μL of each dilution was aseptically spread onto a lysogeny agar plate. The plates were incubated at 37° C. overnight. A total viable count of recoverable E. coli was calculated. This protocol was carried out for each of the following compounds: 1% H2O2+1% KI+1% KSCN (labeled 1 mg SAC), 0.1% H2O2+0.1% KI+0.1% KSCN (0.1 mg SAC), 0.1 mg Gentamycin, 1 mg Gentamycin, 0.01 mg Levofloxacin, 0.1 mg Levofloxacin, 1 mg Levofloxacin, 10 mg Levofloxacin, 0.01 mg Polymyxin B, 0.1 mg Polymyxin B, 1 mg Polymyxin B, 0.1 mg H₂O₂, 1 mg H₂O₂, 10 mg H₂O₂. As shown in FIGS. 5 and 6, the novel iodophor of the invention showed excellent killing efficiency against E. coli ATCC 25922. It showed equivalent performance to several of the antibiotics tested including Levofloxacin and Polymyxin B (at all concentrations). The novel iodophor proved superior to Gentamycin (at both 0.1 mg and 1 mg concentrations) at killing the microorganism. Against P. aeruginosa the novel iodophor showed excellent killing efficiency, greater than 90% killing at all concentrations tested. This is in contrast to all the antibiotics tested, with the exception of Levofloxacin at a concentration of 10 mg. For all other antibiotics and concentrations used, they were unable to kill more than 20% of the bacteria. Levofloxacin at a concentration of 10 mg was efficient at killing P. aeruginosa however the concentration employed (10 mg) is a very high dosage and the upper limit at which it will go into solution. Against both organisms, Hydrogen peroxide was seen to have limited killing efficiency, even at high concentrations (10 mg) employed.

This example therefore shows the potential of the iodophor to be delivered via nebulisation while retaining an extremely high degree of efficacy, particularly against P. aeruginosa a common respiratory pathogen.

### Results

### Minimum Inhibitory Concentrations

The efficacy of the novel 1% (1:1) as produced as described in Example 9 was tested against a bank of microorganisms associated with skin, mastitis and lung infections to determine its activity. To perform such efficacy testing, minimum inhibitory concentrations (MIC) tests were carried out using the micro-broth dilution method wherein doubling dilutions of the compound are added to wells containing the test organism (10⁵⁻⁶ colony forming units ml⁻¹) in a growth medium. The optical density of the well contents are then recorded over 24 hours. An increase in optical density indicates growth of the test organism. If the bacteria failed to grow over 24 hours at 37° C. in a particular well, the concentration therein was deemed inhibitory. The lowest concentration required to inhibit the growth is known as the MIC. For tests involving no growth medium (i.e. water and milk), an aliquot from the well was subcultured to fresh nutrient agar (NA). The corresponding compound concentration was deemed inhibitory if bacteria subsequently failed to grow from the sampled aliquot.

As is clear from Table 1, the iodophor (1%, 1:1) is effective at inhibiting each of the organisms (including antibiotic-resistant organisms) at concentrations of less than 30 mg L⁻¹. This level of activity is approaching antibiotic levels of activity at low concentrations. As a comparison, PVP-I and Lugol's iodine were unsuccessful at eliminating the same bacterial strains below concentrations of 500-1,000 mg L⁻¹. This is due to the deleterious inactivation of the PVP-I or Lugol's-derived iodine by organic material present in the growth medium. Such differences in MICs were noted in various growth media and a milk environment (MIC of <30 mg L⁻¹ for the iodo-thiocyanate iodophor and >500-1000 L⁻¹ PVP-I/Lugol's).

TABLE 1
Minimum Inhibitory Concentration values of the iodo-thiocyanate
iodophor for bacterial strains in Lysogney Broth

| Organism | MIC (mg/L) |
|---|---|
| E. coli ATCC 25922 | 10-20 |
| Pseudomonas aeruginosa PA01 | 10-20 |
| P. aeruginosa PA-29 (antimicrobial tolerant isolate | 10-20 |
| Mc Cay et al., 2010) | |
| P. aeruginosa 9026 (cystic fibrosis isolate, antibiotic | 10-20 |
| tolerant) | |
| P. aeruginosa 9027 (cystic fibrosis isolate, antibiotic | 10-20 |
| tolerant) | |
| P. aeruginosa 9028 (cystic fibrosis isolate, antibiotic | 10-20 |
| tolerant) | |
| P. aeruginosa 9029 (cystic fibrosis isolate, antibiotic | 10-20 |
| tolerant) | |
| Staphylococcus aureus 15676 | 15-30 |
| S. aureus BH1CC (MRSA, oxacillin resistant | 15-30 |
| hospital isolate) | |
| S. eipdermidis 1457 (oxacillin resistant hospital | 15-30 |
| isolate) | |
| S. epidermidis C57 | 15-30 |
| Micrococcus luteus | 10-20 |
| Dermacoccus nishinomiyaensis | 10-20 |
| Strep. agalactiae (isolate from mastitic ruminant) | 5-10 |
| Strep. dysgalactiae (isolate from mastitic ruminant) | 5-10 |
| Strep. uberis (isolate from mastitic ruminant) | 5-10 |
| Mycobacterium bovis | <200 mg/l |
| Cronobacter sakazakii | <200 mg/l |
| Chlamydia trachomatis | <200 mg/l |
| Neisseria gonorrhoeae | <200 mg/l |
| Grp. B. streptococcus | <200 mg/l |
| Mycobacterium tuberculosis | <200 mg/l |
| Mycoplasma pneumoniae | <200 mg/l |
| Mycobacterium bovis | <200 mg/l |
| C. pneumoniae | <200 mg/l |
| Plesiomonas | <200 mg/l |
| Moraxella | <200 mg/l |
| trueperella pyogenes | <200 mg/l |
| Legionella micdadei | <200 mg/l |
| Legionella pneumophilia | <200 mg/l |
| Bordetella pertussis | <200 mg/l |
| Bordetella para-pertussis | <200 mg/l |
| Gardnerella vaginalis | <200 mg/l |
| Trichomonas vaginalis | <200 mg/l |
| Borelia burgdorfi | <200 mg/l |
| C difficile | <200 mg/l |
| bordetella | <200 mg/l |
| Campylobacter | <200 mg/l |
| fusobacterium necrophorum | <200 mg/l |
| staphylococcus hyicus | <200 mg/l |
| arcanobacter pyogenes | <200 mg/l |
| Enterococcus faecalis | <200 mg/l |
| Enterococcus faecium | <200 mg/l |
| Streptococcus pneumoniae | <200 mg/l |
| Streptococcus agalactiae | <200 mg/l |
| Streptococcus pyogenes | <200 mg/l |
| Streptococcus mitis | <200 mg/l |
| Streptococcus viridans | <200 mg/l |
| legionella | <200 mg/l |
| streptococcus zooepidemicus | <200 mg/l |
| Streptococcus mutans | <200 mg/l |
| Klebsiella pneumoniae | <200 mg/l |
| klebsiella oxytoca | <200 mg/l |
| Serratia marcescens | <200 mg/l |
| enterobacter cloacae | <200 mg/l |
| enterobacter aerogenes | <200 mg/l |
| proteus mirabilis | <200 mg/l |
| haemophilus | <200 mg/l |
| acinetobacter baumanaii | <200 mg/l |
| stenotrophomonas maltophilia | <200 mg/l |
| micrococcus | <200 mg/l |
| bacillius | <200 mg/l |
| listeria | <200 mg/l |
| listeria | <200 mg/l |
| lactobacillus | <200 mg/l |
| burkholderis | <200 mg/l |
| comamonas | <200 mg/l |
| acidovorax | <200 mg/l |
| salmonella | <200 mg/l |

Comparative concentrations of 0.01-5 mg L⁻¹ of the novel iodo-thiocyanate iodophor, Lugol's solution and PVP-I were sufficient in killing the same bacterial strains in the absence of any growth medium (i.e. a saline environment), demonstrating that the difference in relevance activity is due to the organic material.

The described compositions have been shown to be bacteriocidal and not bacteriostatic. If an antimicrobial compound is removed from the environment and the bacteria/virus/fungi/yeast once again begin to proliferate, the compound would be described as bacteriostatic (i.e. stopping the cells from growing). If they didn't recover the compound would be classed as bacteriocidal (i.e. killing of cells). In tests with the novel iodophor, viable bacterial cells were not recoverable using 20 mg L⁻¹ in saline for 1 hour, even with the addition of sodium pyruvate to the saline to help bacterial recovery. Using an equivalent concentration (and a further concentration 10 times greater) of hydrogen peroxide alone resulted in the recovery of viable cells, demonstrating that the killing was not peroxide mediated.

Antiviral Activity:

The efficacy of the novel 1% (1:1) as produced as described in Example 9 was tested against a bank of viruses associated with various human and veterinary illnesses to determine its activity. A number of different assays were utilised to determine the potential antiviral activity of the compound. These include: Viral Cytopathic Effects (CPE) assay, whereby the compound is tested on its ability to prevent the target virus from causing viral cytopathic effects in mammalian cell culture. The compound was diluted several times (up to 8 dilutions) with effective antiviral concentration determined by regression analysis. In parallel toxicity of the compound was assayed. A Virus Yield Reduction Assay was also conducted to determine the ability of the compound inhibit virus production in mammalian cell culture. This assay is a two-step assay whereby the virus is first produced in a culture containing the compound at various dilutions, followed by titration of the samples for virus titre by endpoint dilution in a 96-well plate. Effective antiviral concentration determined by regression analysis.

A Virucidal Assay is also carried out—this determines whether the compound can kill, or inactivate, the virus outside of cells i.e. whether through contact with the compound the virus is inactivated and is unable to infect cells. This assay is important in showing compounds with potential in environmental settings. The assay is conducted by incubating the virus with the compound for 1 hour, followed by determining virus titre by endpoint dilution in 96-well plates. Results of these studies are shown in the table below:

| Organism | MIC (mg/L) |
|---|---|
| bovine respiratory synctial virus (BRSV) | <200-300 |
| bovine herpesvirus type 1 (BHV-1) | <200-300 |
| bovine parainfluenza virus type 3 (BPIV3) | <200-300 |
| bovine viral diarrhoea virus (BVDv) | <200-300 |
| parvo virus | <200-300 |
| parainfluenza | <200-300 |
| orthomyxovirus equine influenza A type 2 | <200-300 |
| equine-1 (H7N7) virus | <200-300 |
| equine-2 (H3N8) virus | <200-300 |
| Herpes simplex virus-1 | <200-300 |
| Herpes simplex virus-2 | <200-300 |
| Norovirus | <200-300 |
| Adenovirus | <200-300 |
| Poliovirus (types 1 & 3) | <200-300 |
| Rotavirus | <200-300 |
| Coxsackie virus | <200-300 |
| Rhinovirus | <200-300 |
| Rubella virus | <200-300 |
| Measles virus | <200-300 |
| Influenza virus | <200-300 |
| HIV | <200-300 |
| Mumps virus | <200-300 |

The efficacy of the novel 1% (1:1) as produced as described in Example 9 was tested against a bank of yeasts, moulds and fungi associated with various human and veterinary illnesses to determine its activity. Susceptibility testing was carried out according to a variety of protocols developed and approved by the Clinical Laboratory Standards Institute (CLSI), namely, CLSI M27-A2 for susceptibility testing of yeasts; CLSI M44-A for susceptibility testing of yeasts by disk diffusion; CLSI M38-A for susceptibility testing of moulds. Results from these studies are presented in the table below:

| Organism | MIC (mg/L) |
|---|---|
| Trichophyton verrucosum | <200 mg/l |
| Epidermophyton floccosum | <200 mg/l |
| Trichophyton rubrum | <200 mg/l |
| Trichophyton interdigitale | <200 mg/l |
| Trichophyton tonsurans | <200 mg/l |
| Trichophyton mentagrophytes | <200 mg/l |
| Microsporum canis | <200 mg/l |
| Microsporum gypseum | <200 mg/l |
| Candida | <200 mg/l |
| Aspergillus | <200 mg/l |

Similar MICs were obtained for protozoa.

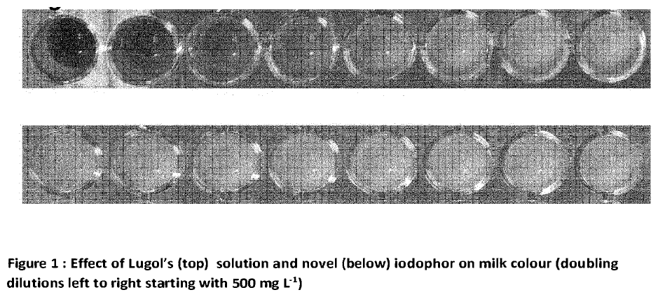

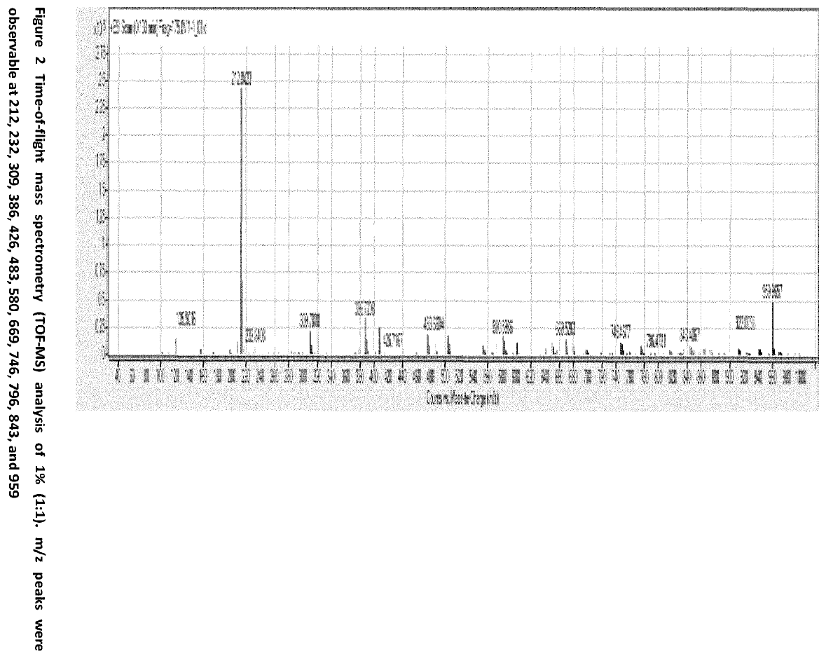

A further benefit of the novel iodophor is the relative lack of staining and colour associated with its use. Lugol's is preferable to PVP-I in terms of activity exerted in a milk environment (though about 40-fold less active than the novel iodophor). However, the concentrations required to kill the bacteria also led to a change in the appearance and colour of the milk. This is not the case with the novel iodophor. FIG. 1 demonstrates the colour change in milk containing 500, 250, 125, 62.5, 31.25, 15.625, 7.8125, 3.9, and 1.95 mg L⁻¹ of each compound (left to right). It is clear that the Lugol's iodine brings about an orange and even brown colouration (down to and including at >10 mg L⁻¹) whereas the novel iodophor does not, even at 500 mg L⁻¹. This would be important when using appropriate dose concentration for mastitis or treatment of contaminated milk supplies.

A 1% (1:1) sample was examined using Time of Flight mass spectrometry (TOF) to identify the complexes responsible for antimicrobial activity. A number of peaks (indicating their mass to charge ratio—m/z) of interest were identified. Peaks at m/z 121 and 922 correspond to internal calibrants. This demonstrates that there was more than one iodo-thiocyanate complex formed during the reaction. A number of larger molecules formed as more iodine atoms attach and allow the attachment of further cyanogen molecules to attach. Peaks were observed at m/z of 212, 309, 386, 483. These m/z values would equate to a number of compounds such as ICNH(SCN), I₂SCN, I₂OH(SCN)₂, and I₂SCHN₂O. Each of these structures is an iodo-thiocyanate complex with minor substitutions and additions. Weaker peaks were identified at m/z ratios of 580, 669, and 746, and 959 indicating more iodine atoms and cyanogen molecules attaching, producing even larger complexes.

To demonstrate that further alternative oxidising agents can be employed to produce the compound instead of hydrogen peroxide, potassium permanganate was used in a disc diffusion assay; small concentrations of the components (100 μg thiocyanate/iodide/permanganate in 10 μl water) were applied to a paper disc on top of lysogeny agar freshly seeded with 10⁵ cfu/ml of E. coli ATCC 25922. Plates are incubated overnight at 37° C. and the resulting zones of inhibition are measured. A large zone indicates strong antimicrobial activity. No zone indicates a lack of perceivable antimicrobial activity. Results from the tests are presented in Table 2. Permanganate can itself exert an antimicrobial effect, but only in the absence of organic material. Therefore, any antimicrobial activity noted in samples containing permanganate is as a result of its oxidation of the thiocyanate and iodide to produce the novel iodophor. It is evident that on mixing the three compounds, strong activity was noted, but only when all three were mixed. It is clearly demonstrated therefore that alternative oxidising agents other than peroxide are capable of producing the iodophor.

TABLE 2
Demonstration of iodophor production by permanganate-mediated
oxidation

| Solution | Zone of inhibition |
|---|---|
| 375 μg KMnO₄ | No zone |
| 375 μg KMnO₄ + 375 μg KSCN | No zone |
| 375 μg KI +375 μg KSCN | No zone |
| 375 μg KMnO₄ + 375 μg KI + 375 μg KSCN | 34 mm |

1.-22. (canceled)

23. A method of killing microorganisms, the method comprising subjecting the microorganisms to an iodophor-producing composition consisting essentially of:
(a) a source of iodide/iodate ions, a source of thiocyanate ions and an oxidizing agent, which are capable of reacting in situ or ex situ to produce an iodophor,
wherein the source of iodide/iodate ions is selected from potassium iodide, sodium iodide, lithium iodide, cesium iodide, hydrogen iodide, rhodium iodide, potassium iodate and sodium iodate, and
wherein the oxidizing agent is selected from potassium iodide, hydrogen peroxide or potassium permanganate, sodium peroxide, lithium peroxide, peroxide releasing percarbonates, peroxide releasing citric acid or vitamin C, peroxide salts including barium oxide, sodium perborate, a hydrogen peroxide-urea adduct, oxygen releasing pseudo-peroxides including superoxides, dioxygenals, ozones, ozonides, organic peroxides including peroxy acids, acyl halides and aliphatic peroxides; and
(b) an optional solvent.

24. The method according to claim 23, wherein the source of thiocyanate ions is selected from sodium thiocyanate, potassium thiocyanate, lithium thiocyanate, cesium thiocyanate, hydrogen thiocyanate, rhodium thiocyanate, allyl isothiocyanate, and a cyanate compound.

25. The method according to claim 23, wherein the source of iodide/iodate ions is potassium iodide and the oxidizing agent is potassium iodide.

26. The method according to claim 23, wherein the ratio of iodide ions from the source of iodide/iodate ions to the thiocyanate ions is in the range of from 0.1:10-10:1 by weight.

27. The method according to claim 23, wherein the iodophor is an iodo thiocyanate complex selected from one or more of ISCN, I₂SCN, I₂(SCN)₂, IOH(SCN)₂, I₃OH(SCN)₂, I₃OH(SCN)₃, I₄(SCN)₄OH, and I₅(SCN)₅.

28. The method according to claim 23, wherein the iodophor comprises at least one oxygen, at least one iodine and at least one thiocyanogen or cyanate unit.

29. The method according to claim 23, wherein the iodophor comprises the reaction products of the iodophor-producing composition when placed in an aqueous environment.

30. The method according to claim 23, wherein the iodophor-producing composition is formulated in a wash, face-wash, an acne cream, a mouth wash, or a nasal rinse.

31. The method according to claim 23, wherein the method comprises reducing the bacterial contamination of milk or feedstuffs by adding the iodophor-producing composition to the milk or feedstuffs.

32. The method according to claim 23, wherein the iodophor-producing composition is a non-acidic iodophor-producing composition.

33. The method according to claim 23, wherein the iodophor comprises an iodo thiocyanate complex containing an oxygen.

34. The method according to claim 23, wherein the microorganisms are bacteria, fungi, viruses, parasites, yeast and/or protozoa.

35. A method of treating an infection in a subject by administering an inhibitory concentration of an iodophor-producing composition consisting essentially of:
(a) a source of iodide/iodate ions, a source of thiocyanate ions and an oxidizing agent, which are capable of reacting in situ or ex situ to produce an iodophor,
wherein the source of iodide/iodate ions is selected from potassium iodide, sodium iodide, lithium iodide, cesium iodide, hydrogen iodide, rhodium iodide, potassium iodate and sodium iodate, and
wherein the oxidizing agent is selected from potassium iodide, hydrogen peroxide or potassium permanganate, sodium peroxide, lithium peroxide, peroxide releasing percarbonates, peroxide releasing citric acid or vitamin C, peroxide salts including barium oxide, sodium perborate, a hydrogen peroxide-urea adduct, oxygen releasing pseudo-peroxides including superoxides, dioxygenals, ozones, ozonides, organic peroxides including peroxy acids, acyl halides and aliphatic peroxides; and
(b) an optional solvent.

36. The method according to claim 35, wherein the source of thiocyanate ions is selected from sodium thiocyanate, potassium thiocyanate, lithium thiocyanate, cesium thiocyanate, hydrogen thiocyanate, rhodium thiocyanate, allyl isothiocyanate, and a cyanate compound.

37. The method according to claim 35, wherein the source of iodide/iodate ions is potassium iodide and the oxidizing agent is potassium iodide.

38. The method according to claim 35, wherein the ratio of iodide ions from the source of iodide/iodate ions to the thiocyanate ions is in the range of from 0.1:10-10:1 by weight.

39. The method according to claim 35, wherein the iodophor is an iodo thiocyanate complex selected from one or more of ISCN, I₂SCN, I₂(SCN)₂, IOH(SCN)₂, I₃OH(SCN)₂, I₃OH(SCN)₃, I₄(SCN)₄OH, and I₅(SCN)₅.

40. The method according to claim 35, wherein the iodophor comprises at least one oxygen, at least one iodine and at least one thiocyanogen or cyanate unit.

41. The method according to claim 35, wherein the iodophor comprises the reaction products of the iodophor-producing composition when placed in an aqueous environment.

42. The method according to claim 35, wherein the iodophor-producing composition is administered to the subject orally, intravenously, topically or parenterally,
or wherein the iodophor-producing composition is formulated and administered to the subject in a cream, a gel, or a paste.

43. The method according to claim 35, wherein the iodophor-producing composition is formulated in a wash, face-wash, an acne cream, a mouth wash, or a nasal rinse.

44. The method according to claim 35, wherein the method comprises reducing the bacterial load in a mammary gland,
or wherein the iodophor-producing composition is administered to the subject with an intra-mammary device or a dampened bandage.

45. The method according to claim 35, wherein the method is a method of treating an infection of a wound or burn in a subject and the iodophor-producing composition is administered to the subject in the form of a bandage or poultice impregnated with the iodophor-producing composition.

46. The method according to claim 35, wherein the method is a method of treating a bacterial or fungal infection of a human or animal lung in a subject and the iodophor-producing composition is administered to the subject in the form of a nebulised spray.

47. The method according to claim 35, wherein the infection is a mastitis infection and the subject is a ruminant animal.

48. The method according to claim 35, wherein the subject is a mastitic lactating ruminant animal.

49. The method according to claim 35, wherein the iodophor-producing composition is a non-acidic iodophor-producing composition.

50. The method according to claim 35, wherein the iodophor comprises an iodo thiocyanate complex containing an oxygen.

51. The method according to claim 35, wherein the infection is a bacterial infection, fungal infection, viral infection, parasitic infection, yeast infection and/or protozoan infection.
